(19) United States
(12) Patent Application Publication
(10) Pub. No.: US 2021/0172953 A1
(43) Pub. Date: Jun. 10, 2021

(54) CANCER CELL DETECTION AND IMAGING SYSTEM, PROCESS AND PRODUCT

(71) Applicant: MASTER DYNAMIC LIMITED, Hong Kong (CN)

(72) Inventors: Ka Wing CHENG, Hong Kong (CN); Koon Chung HUI, Hong Kong (CN); Ching Tom KONG, Hong Kong (CN)

(73) Assignee: MASTER DYNAMIC LIMITED, Hong Kong (CN)

(21) Appl. No.: 17/254,736

(22) PCT Filed: Jun. 21, 2019
(86) PCT No.: PCT/CN2019/092175

§ 371 Date: Dec. 21, 2020

(30) Foreign Application Priority Data
Jun. 22, 2018 (HK) 18108093.9

(51) Int. Cl. G01N 33/574; G01N 21/64; G01N 33/58

(52) U.S. Cl. CPC G01N 33/574; G01N 2021/6421; G01N 33/587; G01N 21/6489

(57) ABSTRACT

A method of distinguishing cancerous cells and healthy cells of a subject from each other comprises the steps: (i) contacting a region of tissue of a subject suspected of including at least some cancer cells with a plurality of nanodiamonds, wherein the plurality of nanodiamonds comprise a first plurality of conjugates, wherein the conjugates of the first plurality of conjugates consist of first nanodiamonds and one or more cancer cell targeting agents, wherein the first nanodiamonds have a first type of colour center, and a second plurality of conjugates, wherein the conjugates of the second plurality of conjugates consist of second nanodiamonds and one or more healthy cell targeting agents, wherein the second nanodiamonds have a second type of colour center; and (ii) applying light of a first wavelength so as to excite the first type of colour center and applying light of a second wavelength so as to excite the second type of colour center, wherein upon contacting the region of tissue with the plurality of nanodiamonds, cancer cells are adhered to the first plurality of conjugates, and healthy cells are adhered to the second plurality of conjugates; wherein upon applying light to the region of tissue, the colour centers of the nanodiamonds of the first plurality of conjugates adhered to cancer cells emit fluorescence at a first wavelength, and the colour centers of the nanodiamonds of the second plurality of conjugates adhered to healthy cells emit fluorescence at a second wavelength; and wherein the colour contrast between the two wavelengths and the positions of respective conjugates delineate the area of cancer cells and the area of healthy cells from each other.

### TECHNICAL FIELD

The present invention relates to the detection of cancer, in particular the present invention provides a product and process for the detection of cancer.

### BACKGROUND OF THE INVENTION

In cancer therapy, it is necessary to completely remove the tumor tissue inside human body in order to eradicate the cancer from the body of a subject.

However, in order to ensure a complete removal, surgeons typically are required to remove excess tissue surrounding the cancer site, as typically there is little or no information on how the tumor may spread.

Therefore, cancer cells labelling techniques have been developed in this regard. Fluorescence dyes have been widely investigated for the applications on cancer cell labelling. By labelling cancer cells with a fluorescent substance, surgeons can minimize the area of removal of tissue of a subject so as to preserve maximal organ function, while providing more accurate and more complete removal of cancer tissue. Patients can thus benefit by way of a quicker and more complete recovery.

However, most fluorescence dyes are cytotoxic to subjects. Therefore, the tumor tissue labelling by such a technique may induce harm to the subject.

Moreover, in order to view the fluorescence, excitation light is needed to be shined or directed onto the fluorescence dye labelled tissues.

Since most of the fluorescence dyes are organic, they are inherently unstable and easily photobleached by strong excitation light. This greatly limits the lifetime of such fluorescence dyes.

### OBJECT OF THE INVENTION

It is an object of the present invention to provide a product and process for the detection of cancer which overcomes or at least partly ameliorates at least some deficiencies as associated with the prior art.

### SUMMARY OF THE INVENTION

In a first aspect, the present invention provides a method of distinguishing cancerous cells and healthy cells of a subject from each other, said method including the steps of (i) contacting a region of tissue of a subject suspected of including at least some cancer cells with a plurality of nanodiamonds, wherein said plurality of nanodiamonds comprises a first plurality of conjugates, wherein the conjugates of the first plurality of conjugates consist of a nanodiamond and one or more cancer cell targeting agents, wherein the nanodiamonds have a first type of colour center, and a second plurality of conjugates, wherein the conjugates of the second plurality of conjugates consist of a nanodiamond and one or more healthy cell targeting agents, wherein the nanodiamonds have a second type of colour center, (ii) applying light of a first wavelength so as to excite said first type of colour center and applying light of a second wavelength so as to excite said second type of colour center; wherein upon contacting the region of tissue with the plurality of nanodiamonds cancer cells are adhered to with said the first plurality of conjugates, and healthy cells are adhered to with said second plurality of conjugates; wherein upon applying light to said region of tissue, the colour centers of the nanodiamonds of the first plurality of conjugates adhered to cancer cells fluoresce at a first wavelength, and the colour centers of the nanodiamonds of the second plurality of conjugates adhered to healthy cells fluoresce at a second wavelength; and wherein the colour contrast between the first wavelength and the second and position of respective conjugates delineates the area of cancer cells and the area of healthy cells from each other.

The nanodiamonds of one of the pluralities of conjugates may include nitrogen colour centers, and the nanodiamonds of the other plurality of conjugates may include silicon colour centers.

The nanodiamonds of the first conjugates may include nitrogen colour centers with a wavelength of 600 nm and the nanodiamonds of the second may include silicon colour centers with a wavelength of 700 nm, resulting in a 100 nm difference in wavelength and light.

Upon application of strong light source, the nitrogen and the silicon colour centers may be excited so as to provide enhanced delineation between the cancerous cells and the healthy cells, by each marked region of type of cells having a different colour, in accordance with the present invention. The light source may be a single wavelength or broad light light source.

In a second aspect, the present provides a marking product for marking cancerous cells and healthy cells of a subject, said marking product comprising a plurality of nanodiamonds which comprises a first plurality of conjugates, wherein the conjugates of the first plurality of conjugates consist of a nanodiamond and one or more cancer cell targeting agents, wherein the nanodiamonds have a first type of colour center, and a second plurality of conjugates, wherein the conjugates of the second plurality of conjugates consist of a nanodiamond and one or more healthy cell targeting agents, wherein the nanodiamonds have a second type of colour center,

The nanodiamonds of one of the pluralities of conjugates may include nitrogen colour centers, and the nanodiamonds of the other plurality of conjugates may include silicon colour centers.

The nanodiamonds of the first conjugates may include nitrogen colour centers with a wavelength of 600 nm and the nanodiamonds of the second may include silicon colour centers with a wavelength of 700 nm, resulting in a 100 nm difference in wavelength and light.

The marking product is provided as a spray, a cream, a lotion or the like.

In a third aspect, the present invention provides a system for distinguishing cancerous cells and healthy cells of a subject from each other, wherein tissue of a subject suspected of including at least some cancer cells has been contacted with a plurality of nanodiamonds, wherein said plurality of nanodiamonds comprises a first plurality of conjugates wherein the conjugates of the first plurality of conjugates consist of a nanodiamond and one or more cancer cell targeting agents, wherein the nanodiamonds have a first type of colour center, a second plurality of conjugates, and wherein the conjugates of the second plurality of conjugates consist of a nanodiamond and one or more healthy cell targeting agents and wherein the nanodiamonds have a second type of colour center, and wherein upon contacting the region of tissue with the plurality of nanodiamonds cancer cells are adhered to with said the first plurality of conjugates, and healthy cells are adhered to with said second plurality of conjugates, said system including one or more light sources for applying light of a first wavelength and of a second wavelength, wherein upon applying light of said first wavelength and of said second wavelength to said region of tissue, the colour centers of the nanodiamonds of the first plurality of conjugates adhered to cancer cells are excited and fluoresce at a first wavelength, and the colour centers of the nanodiamonds of the second plurality of conjugates adhered to healthy cells are excited and fluoresce at a second wavelength; and wherein the colour contrast between the first wavelength and the second and position of respective conjugates delineates the area of cancer cells and the area of healthy cells from each other.

The nanodiamonds of one of the pluralities of conjugates may include nitrogen colour centers, and the nanodiamonds of the other plurality of conjugates may include silicon colour centers.

The nanodiamonds of the first conjugates may include nitrogen colour centers with a wavelength of 600 nm and the nanodiamonds of the second conjugates may include silicon colour centers with a wavelength of 700 nm, resulting in a 100 nm difference in wavelength and light.

The light source may be a single wavelength or broad light light source.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1 shows a schematic representation of the present invention; and

FIG. 2 shows a schematic representation of a system according to the present invention.

### DETAILED DESCRIPTION OF THE INVENTION AND DRAWINGS

The present inventors have identified shortcomings of the prior art, and upon identification of the problems with the prior art, have provided a product and process for the detection of cancer which overcomes or ameliorates the problems of the prior art.

Within the present invention, nanodiamonds are used for cancer detection due to their high chemical stability and low cytotoxicity. The emitted fluorescence from nanodiamonds is stable with little photobleaching. There have been previous studies on using nanodiamonds for various targeted cancer therapeutic applications.

In accordance with the present invention, there is provided a method of distinguishing cancerous cells and healthy cells of a subject from each other.

In order to effect this method, the following steps are applied:

### Step 1

A region of tissue of a subject suspected of including at least some cancer cells is contacted with a plurality of nanodiamonds.

The plurality of nanodiamonds comprises:
a first plurality of conjugates, wherein the conjugates of the first plurality of conjugates consist of a nanodiamond and one or more cancer cell targeting agents, wherein the nanodiamonds have a first type of colour center, and
a second plurality of conjugates, wherein the conjugates of the second plurality of conjugates consist of a nanodiamond and one or more healthy cell targeting agents, wherein the nanodiamonds have a second type of colour center,

Upon contacting the region of tissue with the plurality of nanodiamonds cancer cells are adhered to with said the first plurality of conjugates, and healthy cells are adhered to with said second plurality of conjugates.

### Step 2

Upon applying light to said region of tissue, the colour centers of the nanodiamonds of the first plurality of conjugates adhered to cancer cells fluoresce at a first wavelength, and the colour centers of the nanodiamonds of the second plurality of conjugates adhered to healthy cells fluoresce at a second wavelength.

The colour contrast between the first wavelength and the second and position of respective conjugates delineates the area of cancer cells and the area of healthy cells from each other.

In accordance with the invention, an important application is to label cancer cells using fluorescence nanodiamonds. Labelled cancer cells can be distinguished from healthy cells so that a surgeon may remove the cancer tissue accurately with minimal removal of healthy tissues in surgical operations.

In order to label cancer cells, fluorescence nanodiamonds have to be conjugated with cancer cell specific targeting agents, such as antibodies, hormones or the like.

The nanodiamonds emit fluorescence when excited by light so that the labelled cancer cells can then be detected at the cancer site.

From the contrast of fluorescence area, a surgeon can identify the cancer tissue in an illuminated area at a surgical site.

Further, the invention also utilises methods for labelling healthy cells whilst keeping cancer cells unlabeled. In order to achieve this, fluorescence nanodiamonds have to be conjugated with healthy cell specific targeting agents. In this way, from the negative contrast of fluorescence area, surgeon can then identify the cancerous tissue in darkened area.

In accordance with the present invention, enhanced contrasting is achieved between cancerous cells and normal or healthy cells at a surgical site.

In order to achieve this as stated above and as claims, a method is provided of using two or more different types of fluorescence nanodiamond complexes for both cancer cell and normal cell detection

By adhering both types of cells with nanodiamond conjugate with different complexes, fluoresce of the nanodiamonds' colour centers allows the cancerous cells to fluoresce at a first wavelength (first colour) and normal cells to fluoresce and a second wavelength (second colour).

This provides much greater peripheral delineation between cancerous cells and normal cells, and is of great importance from a clinical standpoint, for reasons including:
(i) Greater confidence and information as to what tissue is cancerous,
(ii) Better delineation, so as to know which areas of tissue to remove or treat,
(iii) Ensure sufficient cancerous tissue is removed, and
(iv) Limiting the removal of non-cancerous (healthy) tissue.

There are several types of fluorescence nanodiamonds. The differences between them belong to their colour center types, such as nitrogen-vacancy center (NV), nitrogen-vacancy-nitrogen (NVN) center, silicon-vacancy (SiV) center, germanium-vacancy (GeV) center and the like.

These vacancies give fluorescence nanodiamonds the ability to emit fluorescence in different wavelength regions, upon receiving irradiation from an appropriate excitation light source.

Nanodiamonds having surface functional groups, can be linked to targeting agents, such as antibodies, as required. H—C bonds on the surface of H-terminated nanodiamonds allow for nanodiamonds to react like organic compounds. This allows high flexibility in attaching cancer targeting agents on nanodiamonds.

For COOH-terminated or NH₂-terminated nanodiamonds, there are well established methods in conjugation with protein-based targeting agents, such as antibodies.

It is because these targeting agents are protein in nature, there are a lot of amino (—NH₂) and carboxylic (—COON) groups. The amino and carboxylic groups can react with the surface carboxylic and amino groups on nanodiamonds respectively, to form amide bonds. This can be made readily by a reaction with N-hydroxysulfosuccinimide (sulfo-NHS) and 1-ethyl-3-[3-dimethylaminopropyl]carbodiimide hydrochloride (EDC).

Fluorescence nanodiamonds can be conjugated with cancer targeting agents, as in the present invention. This kind of cancer targeting nanodiamonds complex can be applied on tumor affected regions of a subject.

The targeting agents on the nanodiamonds can attach to the receptors or antigens on the surface of cancer cells. By endocytosis, some nanodiamonds may even be engulfed into the cancer cells of the subject. Therefore, only cancer tissues will have fluorescence nanodiamonds remaining on or inside the cancer cells.

Fluorescence nanodiamonds with another type of colour centers can be conjugated with normal cells targeting agents. This kind of normal cells targeting nanodiamonds complex can be applied on tumor affected regions. The targeting agents on the nanodiamonds can attach to the receptors or antigens on the surface of normal cells. By endocytosis, some nanodiamonds may even be engulfed into the normal cells. Therefore, only normal tissues will have fluorescence nanodiamonds remain on or inside the cells.

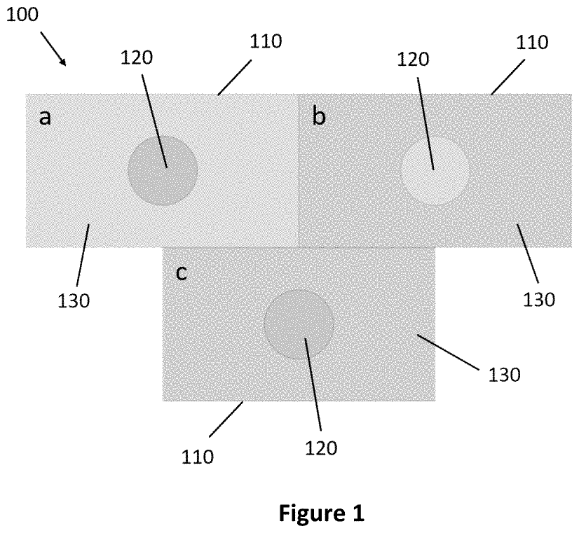

Referring to FIG. 1, there is shown a schematic representation of the present invention 100, and by way of comparison.

Referring to FIG. 1(a), a tissue area 110 of suspected cancer 120 is shown, wherein the suspected cancer 120 is only marked with a first marker, such as nanodiamonds as in the present invention, and upon being illuminated, only the cancer area 120 is shows fluorescence, resulting in uncertainty and poor peripheral delineation between the cancer cell area 120 and the area of normal cells 130.

Referring to FIG. 1(b), the tissue area 130 of normal healthy is only marked with a second marker, such as nanodiamonds as in the present invention, and upon being illuminated, only the normal shows fluorescence of a different wavelength, again resulting in uncertainty and poor peripheral delineation between the cancer cell area 120 and the area of normal cells 130.

As may be understood, in either FIG. 1(a) or (b) unmarked cells may be either cancerous or healthy, and there is uncertainty of which may be which, and the extent of cancer cells.

The present invention overcomes these problems, by having both cancerous and healthy cells marked so as to adhere with appropriate and respective targeting agents, thus marking both cancerous and healthy cells as shown in FIG. 1(c), which may be considered a type of superimposition of FIGS. 1(a) and (b).

This provides advantageously a contrasted and enhanced image combining (a) and (b).

As will be understood, by using different types of fluorescence nanodiamonds for positive contrast and negative contrast imaging respectively as in the present invention, essentially the two contrast methods can be combined together to enhance the imaging contrast by different kinds of fluorescence between cancer cells and healthy cells like in FIG. 1(c).

In accordance with the invention, for example, because of uncontrolled rapid growth in cancer cells, they have much more growth hormones specific receptors. Fluorescence nanodiamonds can be conjugated with these growth hormones, such as epidermal growth factor (EGF), gastrin, etc., to provide positive contrast. On the other hand, cancer cells can lack some receptors that healthy cells have.

Fluorescence nanodiamonds conjugating with specific antibodies for these receptors can give negative contrast imaging on cancer cells. Using nanodiamonds with NV centers for growth hormones for positive imaging and using nanodiamonds with NVN centers for membrane protein, can together give enhanced contrast in imaging. Cancer and healthy cells can then be identified more easily by detecting fluorescence at different wavelength regions.

As will be understood, the present invention may be used either internal or external of the body of subject. The plurality of nanodiamonds containing the first plurality of conjugates and the first plurality of conjugates can be applied to the relevant area of interest of tissue by numerous means or products, for example by way of a spray, a cream, a lotion or the like.

Once the conjugates have adhered to the respective tissue types, excess or non-adhered nanodiamonds may be removed by different means if necessary, such as flushing with physiological fluid, saline or the like, or other methods including air or gas blowing. As will be understood, any such method shall fall within the scope of the invention.

In an example of the invention, the nanodiamonds of the first conjugates may include nitrogen colour centers with a wavelength of 600 nm and the nanodiamonds of the second conjugates may include silicon colour centers with a wavelength of 700 nm, resulting in a 100 nm difference in wavelength and light.

In such an example, a strong light source, such as a single wavelength or broad light may be used to excite the nitrogen and the silicon colour centers, so as to provide enhanced delineation between the cancerous cells and the healthy cells, by each marked region of type of cells having a different colour, in accordance with the present invention.

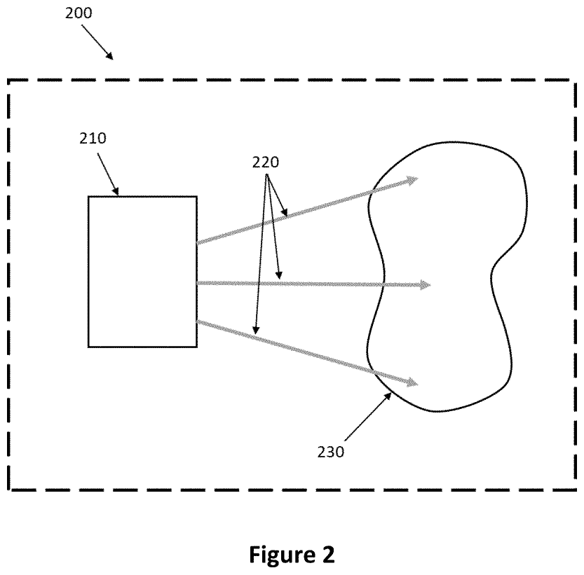

Referring to FIG. 2, there is shown a schematic representation of a system 200 for use in the present invention, as shown and described with reference to FIG. 1.

The system 200 includes one or more light sources 210 for applying light of a first wavelength and of a second wavelength.

Upon applying light 220 of said first wavelength and of said second wavelength to the region of tissue 230, the colour centers of the nanodiamonds of the first plurality of conjugates adhered to cancer cells are excited and fluoresce at a first wavelength, and the colour centers of the nanodiamonds of the second plurality of conjugates adhered to healthy cells are excited and fluoresce at a second wavelength as shown and described with reference to FIG. 1.

As described above, the colour contrast between the first wavelength and the second and position of respective conjugates delineates the area of cancer cells and the area of healthy cells from each other.

The one or more light source is a strong light source, such that as described above upon application of light the nitrogen and the silicon colour centers are excited so as to provide enhanced delineation between the cancerous cells and the healthy cells, by each marked region of type of cells having a different colour, in accordance with the present invention. The light source may be a single wavelength or broad light light source.

1. A method of distinguishing cancerous cells and healthy cells of a subject from each other, said method including the steps of:
contacting a region of tissue of a subject suspected of including at least some cancer cells with a plurality of nanodiamonds, wherein said plurality of nanodiamonds comprises:
a first plurality of conjugates, wherein the conjugates of the first plurality of conjugates consist of a nanodiamond and one or more cancer cell targeting agents, wherein the nanodiamonds have a first type of colour center, and
a second plurality of conjugates, wherein the conjugates of the second plurality of conjugates consist of a nanodiamond and one or more healthy cell targeting agents, wherein the nanodiamonds have a second type of colour center,
(ii) applying light of a first wavelength so as to excite said first type of colour center and applying light of a second wavelength so as to excite said second type of colour center;
wherein upon contacting the region of tissue with the plurality of nanodiamonds cancer cells are adhered to with said the first plurality of conjugates, and healthy cells are adhered to with said second plurality of conjugates;
wherein upon applying light to said region of tissue, the colour centers of the nanodiamonds of the first plurality of conjugates adhered to cancer cells fluoresce at a first wavelength, and the colour centers of the nanodiamonds of the second plurality of conjugates adhered to healthy cells fluoresce at a second wavelength; and
wherein the colour contrast between the first wavelength and the second and position of respective conjugates delineates the area of cancer cells and the area of healthy cells from each other.

2. The method according to claim 1, wherein the nanodiamonds of one of the pluralities of conjugates include nitrogen colour centers, and the nanodiamonds of the other plurality of conjugates include silicon colour centers.

3. The method according to claim 1, wherein the nanodiamonds of the first conjugates include nitrogen colour centers with a wavelength of 600 nm and the nanodiamonds of the second conjugates include silicon colour centers with a wavelength of 700 nm, resulting in a 100 nm difference in wavelength and light.

4. The method according to claim 3, wherein upon application of strong light source, the nitrogen and the silicon colour centers are excited so as to provide enhanced delineation between the cancerous cells and the healthy cells, by each marked region of type of cells having a different colour.

5. The method according to claim 4, wherein the light source is a single wavelength or broad light light source.

6. A marking product for marking cancerous cells and healthy cells of a subject, said marking product comprising a plurality of nanodiamonds which comprises a first plurality of conjugates, wherein the conjugates of the first plurality of conjugates consist of a nanodiamond and one or more cancer cell targeting agents, wherein the nanodiamonds have a first type of colour center, and a second plurality of conjugates, wherein the conjugates of the second plurality of conjugates consist of a nanodiamond and one or more healthy cell targeting agents, wherein the nanodiamonds have a second type of colour center.

7. The marking product according to claim 6, wherein the nanodiamonds of one of the pluralities of conjugates include nitrogen colour centers, and the nanodiamonds of the other plurality of conjugates include silicon colour centers.

8. The marking product according to claim 6, wherein the nanodiamonds of the first conjugates include nitrogen colour centers with a wavelength of 600 nm and the nanodiamonds of the second may include silicon colour centers with a wavelength of 700 nm, resulting in a 100 nm difference in wavelength and light.

9. The marking product according to claim 6, wherein the marking product is provided as a spray, a cream, a lotion or the like.

10. A system for distinguishing cancerous cells and healthy cells of a subject from each other, wherein tissue of a subject suspected of including at least some cancer cells has been contacted with a plurality of nanodiamonds, wherein said plurality of nanodiamonds comprises a first plurality of conjugates wherein the conjugates of the first plurality of conjugates consist of a nanodiamond and one or more cancer cell targeting agents, wherein the nanodiamonds have a first type of colour center, and a second plurality of conjugates, and wherein the conjugates of the second plurality of conjugates consist of a nanodiamond and one or more healthy cell targeting agents and wherein the nanodiamonds have a second type of colour center, and wherein upon contacting the region of tissue with the plurality of nanodiamonds cancer cells are adhered to with said the first plurality of conjugates, and healthy cells are adhered to with said second plurality of conjugates, said system including:
one or more light sources for applying light of a first wavelength and of a second wavelength,
wherein upon applying light of said first wavelength and of said second wavelength to said region of tissue, the colour centers of the nanodiamonds of the first plurality of conjugates adhered to cancer cells are excited and fluoresce at a first wavelength, and the colour centers of the nanodiamonds of the second plurality of conjugates adhered to healthy cells are excited and fluoresce at a second wavelength; and
wherein the colour contrast between the first wavelength and the second and position of respective conjugates delineates the area of cancer cells and the area of healthy cells from each other.

11. The system according to claim 10, wherein the nanodiamonds of one of the pluralities of conjugates include nitrogen colour centers, and the nanodiamonds of the other plurality of conjugates include silicon colour centers.

12. The system according to claim 10, wherein the nanodiamonds of the first conjugates include nitrogen colour centers with a wavelength of 600 nm and the nanodiamonds of the second conjugates include silicon colour centers with a wavelength of 700 nm, resulting in a 100 nm difference in wavelength and light.

13. The system according to claim 10, wherein the one or more light source is a strong light source, and wherein upon application of light the nitrogen and the silicon colour centers are excited so as to provide enhanced delineation between the cancerous cells and the healthy cells, by each marked region of type of cells having a different colour.

14. The system according to claim 10, wherein the light source is a single wavelength or broad light source.
